(19) United States
(12) Patent Application Publication
(10) Pub. No.: US 2013/0226118 A1
(43) Pub. Date: Aug. 29, 2013

(54) DISPOSABLE URINE COLLECTION DEVICE AND URINE COLLECTION APPARATUS

(71) Applicant: Elmer G. ENRIQUEZ, College Point, NY (US)

(72) Inventor: Elmer G. ENRIQUEZ, College Point, NY (US)

(21) Appl. No.: 13/403,306

(22) Filed: Feb. 23, 2012

(51) Int. Cl. A61F 5/451

(52) U.S. Cl. 604/347

(57) ABSTRACT

A disposable urine collection device includes a flexible tubular body having opposite first and second open ends, and a flexible collection bag for collecting and storing therein urine deposited into the first open end of the tubular body. The collection bag is secured to the tubular body and completely encloses the second open end of the tubular body to place the first open end in fluid communication with the interior of the collection bag. Urine deposited into the first open end of the tubular body flows into the collection bag for storage therein. A urine collection apparatus incorporates the disposable urine collection device with a disposable diaper-like garment for use by handicapped or bedridden patients.

### BACKGROUND OF THE INVENTION

1. Field of the Invention

The present invention relates generally to urinal and incontinence control for human beings. More specifically, the invention relates to a disposable urine collection device that allows for the collection of urine, and to a urine collection apparatus that incorporates the disposable urine collection device with a disposable diaper-like garment.

2. Background Information

A significant number or even a majority of patients admitted to hospitals have some form of urinary incontinence or bed wetting for a variety of medical reasons. For this purpose, hospitals, nursing homes and rehabilitation facilities or institutions have employed various types of urinal and incontinence devices for patients, especially those who are bedridden and handicapped and suffering from various stages of decubitus ulcer or bedsores developing in the skin that is always wet from urine compounded by constant pressure over bony prominence in the buttocks region.

Among these prior art devices, a catheter is used to address urinary incontinence, especially for patients suffering from decubitus ulcers in the skin of the buttocks region. Catherization is considered a radical and invasive procedure requiring experienced professional medical staff for the insertion and removal of the catheter utilizing strict sterile techniques. Catherization is also uncomfortable and can be a source of infection, such as urinary tract infections, particularly when used for long periods of time. For example, with catheterization of male patients, the penis and its urethral opening is completely exposed to gross contamination when such patients suffer from fecal incontinence. Furthermore, catherization is particularly an uncomfortable and unnecessary process if the patient is conscious and in control of its bodily functions.

Disposable diapers are also currently used routinely for all incontinent patients whether in hospitals or at home settings. Despite current improvements in diaper products in connection with absorptivity and air permeability, infections of skin or urinary system can not be precluded completely. One main reason for this problem is that a caregiver is unable to check a patient's fecal status at short intervals. As a result, a gross fecal and urine contamination of the disposable diaper creates an undesirably damp, muggy and foul smelling environment which overflows and contaminates the entire patient's bed. This creates an environment for the development of infections of the skin and most importantly ascending urinary tract infections through the patient's penile opening. If cleaning up is neglected and not done on time as necessary, decubitus ulcers or bed sores will eventually develop in the skin over the bony prominence of the buttocks region.

Other incontinence devices available in the market are full of confusing connections and difficult attachments that are literally impractical and time consuming for routine use in hospitals or in other medical facilities or even at home. Some of these known devices even cause allergic skin reactions due to the use of latex materials.

Conventional incontinence devices also find use with paraplegics and other disabled persons who desire mobility. However, such conventional incontinence devices include difficulties in maintaining the devices on the user over a period of time, and the tendency of the devices to leak.

Thus the foregoing conventional urinal and incontinence devices suffer from various disadvantages and problems, including discomfort, possible infections, leakage, complicated constructions, and complicated steps in connection with application and removal of such devices. Furthermore, in the particular case of a bedridden, substantially immobile patient, one of the concerns of the caregivers is the dignity of the person thus affected. In the case of disposable diapers and bedpans, these are particularly embarrassing for both the patient and the caregiver, and are readily apparent to visitors and passers-by so as to amplify the embarrassment and ensuing psychological stress.

The present invention provides an improved disposable urine collection device and an improved urine collection apparatus that incorporates the disposable urine collection device with a disposable diaper-like garment which obviate the disadvantages and limitations of the prior art devices and apparatuses in this field.

### SUMMARY OF THE INVENTION

It is an object of the present invention to provide a disposable urine collection device which can be worn in a non-intrusive manner and without discomfort and is easy to use.

Another object of the present invention is to provide a disposable urine collection device which can be positioned about the male or female genitalia for collecting urine without unintended leakage.

Another object of the present invention is to provide a disposable urine collection device that rids a bedridden patient of the embarrassment, danger and discomfort of catherization and bedpans in the collection of urine.

It is a further object of the present invention to provide a disposable urine collection device that is not visible to visitors and passers-by when worn by the user and prior, during and after use of the device.

A further object of the present invention is to provide a disposable urine collection device that minimizes infections by protecting the user's genitals by isolating them from surrounding soiled areas, thereby facilitating cleaning of patients by cleaning the soiled surrounding areas first before disposing of the disposable urine collection device after use.

A further object of the present invention is to provide a disposable urine collection device which can be readily applied externally in a non-invasive manner and does not require application under strict sterile conditions, and which can be readily removed and discarded after use.

It is yet a further object of the present invention to provide a disposable urine collection device which is made of materials that will not cause allergic reactions.

Yet a further object of the present invention is to provide a disposable urine collection device that is completely hygienic due to its disposable construction providing for one time use only.

It is another object of the present invention to provide a disposable urine collection device that does not occupy a lot of space and is flexible and compressible yet retains its structural integrity during use to allow urine to flow into the device for collection and subsequent disposal.

Another object of the present invention is to provide a disposable urine collection device that is lightweight and soft feeling to skin contact in very sensitive areas such that patients wearing the device will hardly notice its presence.

It is still another object of the present invention to provide a disposable urine collection device that may be provided in various sizes to accommodate various sizes of male penile organs.

Still another object of the present invention is to provide a disposable urine collection device that is inexpensive to manufacture and thus may be widely provided at low cost to the public.

Yet a further object of the present invention is to provide a urine collection apparatus that incorporates the disposable urine collection device with a disposable diaper-like garment.

The foregoing and other objects of the present invention are carried out by a disposable urine collection device comprising a flexible tubular body having opposite first and second open ends, and a flexible collection bag for collecting and storing therein urine deposited into the first open end of the tubular body. The collection bag is secured to the tubular body and completely encloses the second open end of the tubular body to place the first open end in fluid communication with the interior of the collection bag so that urine deposited into the first open end of the tubular body flows into the collection bag for storage therein.

In one embodiment, the tubular body is in the shape of a cone having a first end with a first diameter and corresponding to the first open end and having a second end with a second diameter larger than the first diameter and corresponding to the second open end. In another embodiment, the tubular body is in the shape of a cone having a first end with a first diameter and corresponding to the first open end and having a second end with a second diameter smaller than the first diameter and corresponding to the second open end.

In one embodiment, the collection bag covers at least a portion of an exterior peripheral surface the tubular body. In another embodiment, the collection bag covers the entire exterior peripheral side surface of the tubular body, is folded over and extends into the first open end, and is secured to an interior surface of the tubular body. In preferred embodiments, the tubular body is made of a compressible foam material.

In another aspect, the present invention provides a urine collection apparatus comprising a disposable diaper, a compressible tubular body, and a flexible collection bag. The disposable diaper has a main diaper body with an anterior fold portion and a posterior fold portion, the anterior fold portion having an opening at a midline section thereof communicating the interior with the exterior of the diaper in an assembled state of the disposable diaper. The tubular body has opposite first and second open ends and is configured for placement in the interior of the diaper adjacent the opening of the anterior fold portion. The collection bag is connected to the tubular body so that the first open end of the tubular body is in fluid communication with the interior of the collection bag. The collection bag is configured to be passed through the opening of the anterior fold portion of the diaper from the interior to the exterior of the diaper for collecting and storing therein urine deposited into the first open end of the tubular body.

### BRIEF DESCRIPTION OF THE DRAWINGS

The foregoing summary, as well as the following detailed description of preferred embodiments of the invention, will be better understood when read in conjunction with the accompanying drawings. For the purpose of illustrating the invention, there is shown in the drawings embodiments which are presently preferred. It should be understood, however, that the invention is not limited to the precise arrangement and instrumentalities shown. In the drawings:

FIG. 1 is a perspective top view of a disposable urine collection device according to a first embodiment of the present invention, with the collection bag shown removed from the interior to the tubular body and in an unfolded state ready for use;

FIG. 2 is an exploded view of the disposable urine collection device shown in FIG. 1;

FIG. 3 is a perspective side view of the disposable urine collection device in FIG. 1, with the collection bag stored in the tubular body;

FIG. 4 is a front elevation view showing the disposable urine collection device being worn by a user and ready for use to collect urine;

FIG. 5 is a front elevation view showing the disposable urine collection device after the urine has been collected and stored in the collection bag and ready for disposal.

FIG. 6 is a top view of a disposable urine collection device according to a second embodiment of the present invention;

FIG. 7 is an exploded view of the disposable urine collection device shown in FIG. 6;

FIG. 8 is a perspective view illustrating a urine collection apparatus according to an embodiment of the present invention showing the disposable urine collection device of FIG. 1 and a diaper-like garment in an unassembled state;

FIG. 9 is a perspective view of the urine collection apparatus in FIG. 8 showing the diaper-like garment and disposable urine collection device in a state of being assembled together in preparation for use;

FIG. 10 is a perspective view of the urine collection apparatus of FIG. 9 with the diaper-like garment shown in an open state to illustrate the positioning of the tubular body of the disposable urine collection device relative the inner surface of the diaper-like garment; and

FIG. 11 is a partial side view of the urine collection apparatus of FIGS. 8-10 in a state of being worn by a user ready for use to collect urine.

### DETAILED DESCRIPTION OF THE PREFERRED EMBODIMENTS

While this invention is susceptible of embodiments in many different forms, this specification and the accompanying drawings disclose some examples of the use of the invention. The invention is not intended to be limited to the embodiments so described, and the scope of the invention will be pointed out in the appended claims.

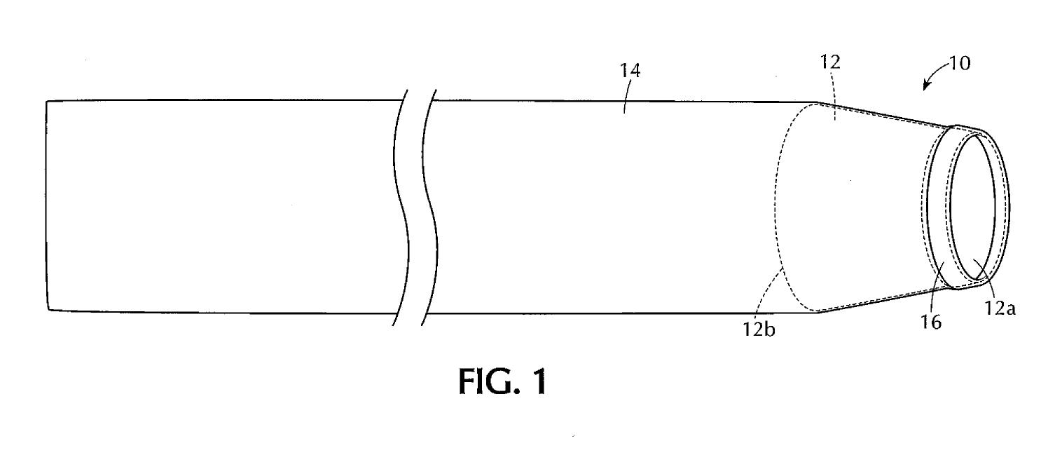

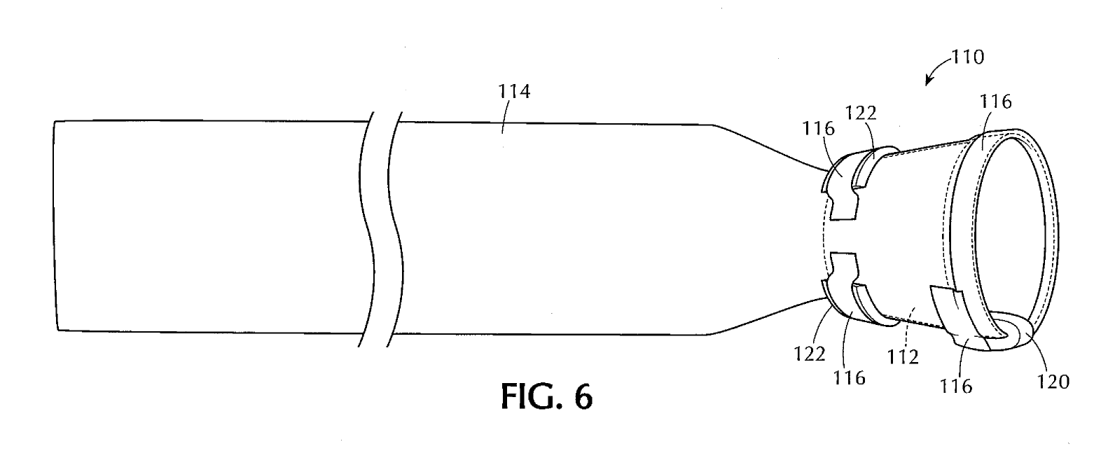

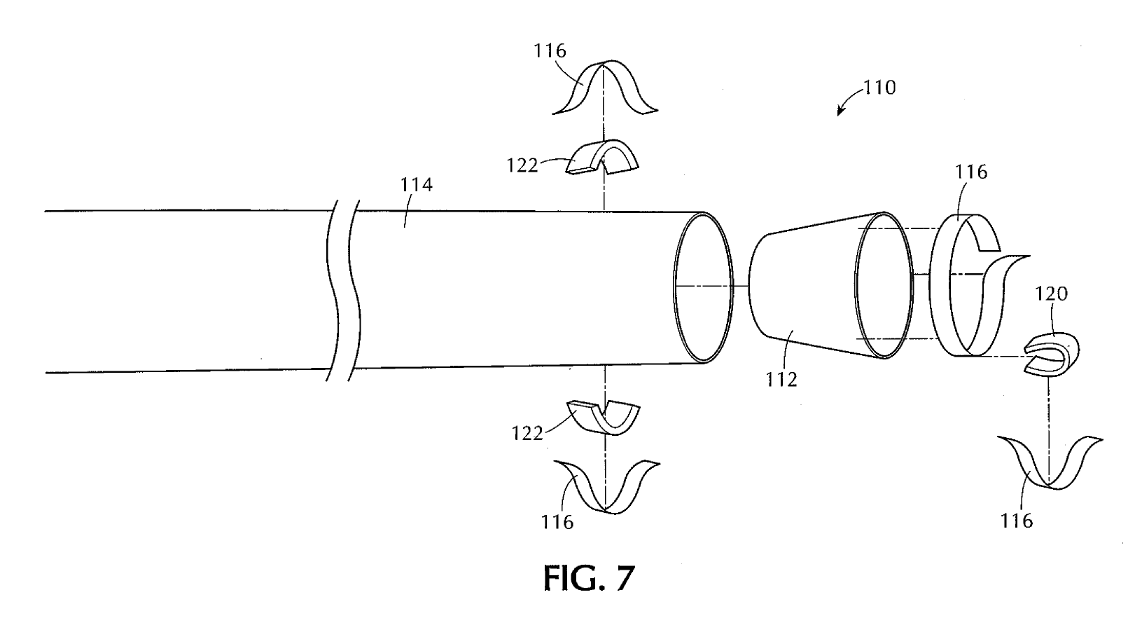

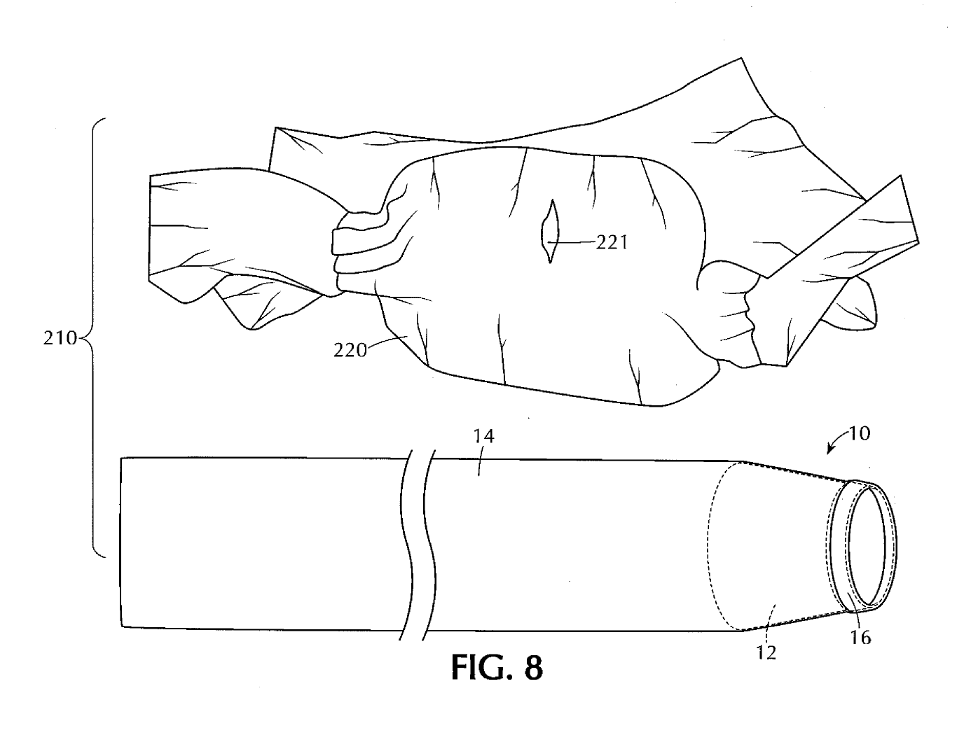

Referring now to the drawings in detail, wherein like numerals are used to indicate like elements throughout, there is shown in FIGS. 1 to 5 a disposable urine collection device, generally designated at 10, according to a first embodiment of the present invention. The disposable urine collection device 10 is configured for urination relief by males, with or without bladder control problems. As further described below, FIGS. 6-7 illustrate another embodiment of the disposable urine collection device configured for urination relief by females, with or without bladder control problems.

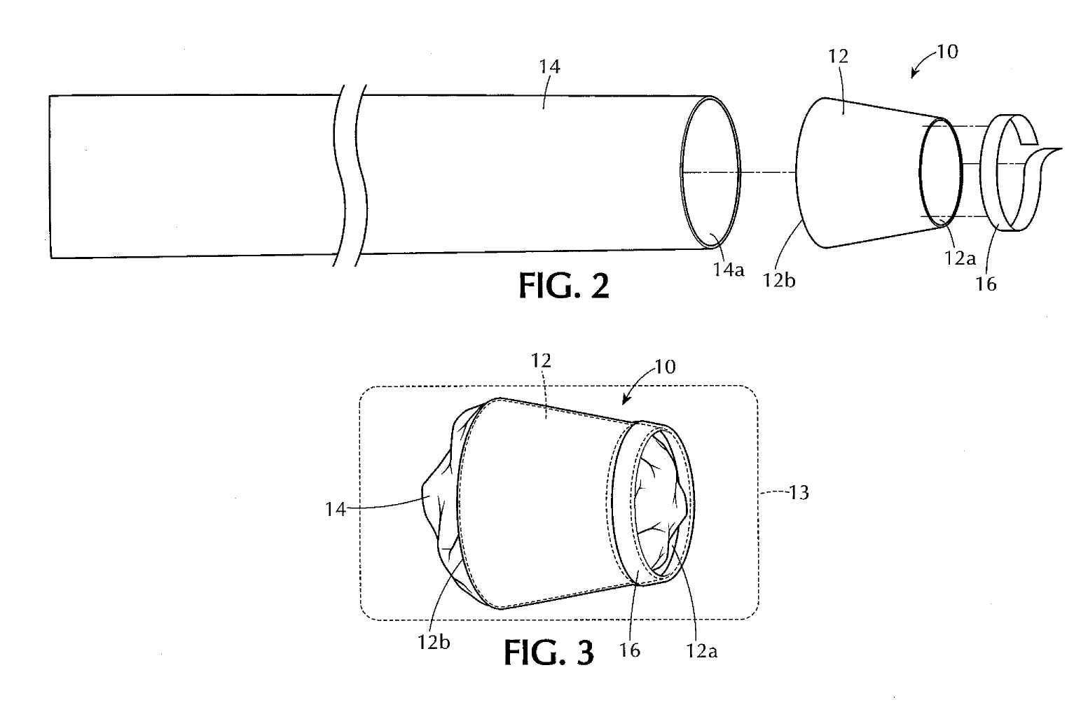

FIG. 1 is a perspective top view of the disposable urine collection device 10 ready for use, and FIG. 2 is an exploded view of the disposable urine collection device in FIG. 1. The disposable urine collection device 10 is formed of a compressible tubular body 12 and a leak-proof, flexible collection bag 14 connected to the tubular body 12 and adapted to receive and hold urine via the tubular body 12. The tubular body 12 has opposite first and second open ends 12a, 12b, and the collection bag 14 is securely attached to the tubular body 12 so as to completely enclose and cover in a liquid-proof manner the second open end 12b, but not the first open end 12a, of the tubular body 12. In use, the first open end 12a of the tubular body 12 is configured to receive the user's penis so that the user is able to urinate into the tubular body 12 which functions to direct and disperse the urine within the collection bag 14 for storage of the urine therein. The diameter of the first open end 12a of the tubular body 12 may be dimensioned in various sizes, including small, medium and large, to accommodate user's of various ages and having various sizes of male genitalia for close fitting.

FIG. 1 depicts the collection bag 14 removed from the interior of the tubular body 12 and in an unfolded state ready for use. As further described below with reference to FIG. 3, the collection bag 14 may be folded and stored within the tubular body 12 so that the entire disposable urine collection device 10 may be conveniently stored and carried in a compact manner (e.g., in the pocket of a garment or a purse) for use when needed.

As depicted in FIG. 1, the collection bag 14 is fitted around and covers the entire exterior peripheral side surface of the tubular body 12 with a portion of the open end 14a of the collection bag 14 being partially folded into the first open end 12a of the tubular body 12 and securely attached to an interior surface of the tubular body 12. In one modified form (not shown) of the embodiment, the collection bag 14 extends into and covers the entire inner peripheral surface of the tubular body 12 and is secured directly to the inner peripheral surface of the tubular body 12 or to an inner portion of the collection bag itself. In another modified form (not shown), the collection bag 14 is secured to an end portion of the tubular body 12 proximate the first open end 12a without being folded into and secured to the interior surface of the tubular body 12. In another modified form (not shown), the collection bag 14 covers only a portion of the exterior peripheral side surface of the tubular body 12. In yet another modified form (not shown), the collection bag 14 does not cover any portion of the exterior peripheral side surface and inner peripheral surface of the tubular body 12, but is instead secured to an end portion of the tubular body 12 at the second open end 12b. It is appreciated that the embodiment shown in FIG. 1 or any of the foregoing modified forms of the embodiment may be adopted for the disposable urine collection device 10 so long as the collection bag 14 completely encloses the second open end 12b of the tubular body 12 in a leak-proof manner such that urine flowing into the tubular body flows into and is received by the collection bag 14 without leaking to the exterior of the disposable urine collection device 10.

By the foregoing construction, the tubular body 12 functions both as an anchor to attach the collection bag 14 and also to enclose the user's penis and to direct and disperse the urine within the collection bag 14 which stores the urine. The disposable urine collection device 10 helps and protects the penis by enclosing it inside the tubular body 12, thereby isolating the penis from any surrounding soiled areas, such as in the event that the device 10 is used by a patient suffering from fecal incontinence. The device 10 also facilitates cleaning of the patient by allowing soiled surrounding areas of the patient's genitalia to be cleaned first before removal and disposal of the device 10. Accordingly, the device 10 minimizes ascending urinary tract infections by isolating the penis inside the tubular body 12 and facilitates cleaning of soiled areas of a patient.

As depicted in FIGS. 1-2, the collection bag 14 is secured to the tubular body 12 using an adhesive tape 16. However, other means for securing the collection bag 14 to the tubular body 12 are suitable, including various types of non-toxic adhesives and/or one or more elastic rings, so long as such securing means provides for a secure attachment of the collection bag 14 to the tubular body 12 so that the collection bag 14 completely encloses the second open end 12b in a liquid-proof manner and does not become detached from the tubular body 12 during use of the disposable urine collection device 10 or during storage of the collection bag 14 inside of the tubular main body 12 as further described below.

The tubular body 12 is preferably in the shape of a cone with its exterior peripheral side surface converging toward the first open end 12a (small opening of the cone) and diverging toward the second open end 12b (large opening of the cone). By forming it in the shape of a cone, the tubular body 12 can be easily positioned and manipulated during use and facilitates dispersion of the urine flowing therein into the collection bag 14.

The tubular body 12 is preferably made of a disposable, liquid-proof compressible plastic material, such as polystyrene foam (commonly referred to by its trade name STYROFOAM® manufactured by The Dow Chemical Company, Delaware). Polystyrene foam is soft and can be easily molded back into its original shape after being compressed, even when wet. Furthermore, when crumpled polystyrene foam does not form sharp edges which may injure the sensitive skin of the user's genitalia. Certain other plastic materials, on the other hand, are usually rigid or hard feeling to skin contact, and when they break even by accident, they form sharp edges at the breaking point which may cause injury to the sensitive skin of the genitalia. Certain paper-based materials are also suitable for the tubular body 12 so long as they are liquid-proof and will not crumple easily when wet, and have the ability to regain back their shape upon compression and will not become loose and easily dislodged.

The collection bag 14 is preferably a liquid-proof, disposable plastic bag (e.g., a clear and scented or unscented plastic material) made of a thin, flexible plastic film which can be folded several times to reduce its size to facilitate storage thereof into the tubular body 12 as described below. The plastic bag may be provided of various suitable liquid storing capacities. The plastic material for the bag may be polyethylene, or any type of biodegradable material. A disposable plastic bag which may be utilized for the collection bag 14 is sold under the trademark RUFFIE® by Poly-tech, Inc., Minnesota.

An exemplary embodiment of a method of assembling the disposable urine collection device 10 according to the present invention is described below.

In the exemplary embodiment described above with reference to FIGS. 1-2, the disposable urine collection device 10 is formed of only the following three components: the tubular body 12, collection bag 14, and adhesive tape 16 as just an example of means for securing the collection bag 14 to the tubular body 12. A commercially available disposable plastic bag (e.g., RUFFIES®) as described above is selected as the collection bag 14. The tubular body 12 is made of polystyrene foam that is formed into a conical shape with first and second open end portions 12a, 12b utilizing any suitable, known fabrication technique. For example, the tubular body 12 can be formed using a commercially available STYROFOAM° cup (e.g., 4 oz cup) from which the bottom at the closed end is removed (e.g., cut out) to provide the first open end 12a and with the open end of the cup serving as the second open end 12b.

To assemble the disposable urine collection device 10 illustrated in FIGS. 1-5, the collection bag 14 is placed over the exterior peripheral side surface of the tubular body 12, such as by inserting the tubular body into open end 14a of the collection bag 14 until the second open end 12b of the tubular body is completely inside of the collection bag 14 (i.e., the second open end 12b of the tubular body 12 is completely covered by the collection bag 14 as shown in FIG. 1). The placement of the collection bag 14 over the exterior peripheral side surface of the tubular body 12 is continued until a terminal end portion of the collection bag 14 that forms the open end 14a extends about one-half inch from the first open end portion 12a of the tubular body 12. This extended portion of the collection bag 14 is then folded into the first open end 12a of the tubular body 12 and secured to an inner surface portion of the tubular body using the adhesive tape 16 as shown in FIGS. 1-2.

In an alternative embodiment (not shown), the extended portion of the collection bag 14 that extends from the first open end 12a of the tubular body 12 is of a sufficient length so as to allow the extended portion of the collection bag to cover the entire inner peripheral side surface of the tubular body. The extended portion of the collection bag 14 can then be secured directly to the interior peripheral surface of the tubular body 12 or to an inner portion of the collection bag 14 ahead of the second open end 12b of the tubular body. By this construction, the collection bag completely encloses the exterior peripheral side surface and the interior peripheral surface of the tubular body 12 so that urine flowing into the collection bag 14 through the tubular body 12 does not directly contact the inner peripheral surface of the tubular body.

In another alternative embodiment (not shown), two of the tubular bodies 12 (e.g., STYROFOAM® cups) may be sleeved and secured together prior to assembling the collection bag 14 as described above in order to provide an added feeling of firmness and to reduce the overall compressibility of the resulting tubular body of the disposable urine collection device.

FIG. 3 is a perspective side view of the disposable urine collection device 10 in which the collection bag 14, when empty, is stored in the tubular body 12. A portion of the collection bag 14 extends out of the second open end 12b (left side of the tubular body in the view shown in FIG. 3) only for the purpose of illustrating how the collection bag is stored inside of the tubular body through the second open end 12b. The tubular body 12 and collection bag 14 are configured so that in a fully stored state, the collection bag 14 is completely disposed and reliably retained within the tubular body 12 in a manner in which it does not extend out of the first and second open ends 12a, 12b of the tubular body. By this construction, the disposable urine collection device 10 has high portability and is easy to carry and store in a manner ready for use. Additionally, in the stored state shown in FIG. 3, the flexible and compressible nature of the materials forming the collection bag 14 and the tubular body 12 allow these components to be easily folded and compressed, respectively, for storage and carrying. When it is then desired to use the disposable urine collection device 10, the compressed tubular body 12 can be easily brought back to its tubular shape and the collection bag 14 can be removed from interior of the tubular body 12 and unfolded into the configuration ready for use as shown in FIG. 1.

In FIG. 3, the dashed line denoted by reference number 13 is a general representation of a storage bag configured to store the disposable urine collection device 10. The storage bag 13 may be any commercially available disposable sterile bag made of plastic, paper or other suitable material and provided with a tamper-evident indicator that safeguards against tampering with the disposable urine collection device 10. For example, the tamper-evident indicator may be in the form of a tamper-evident closure for the disposable sterile bag 13 and/or some type of indicia incorporated to the disposable sterile bag for readily indicating tampering with the disposable urine collection device 10. The disposable sterile bag 13 allows the user, a healthcare professional or any other person handling the disposable sterile bag 13 containing the disposable urine collection device 10 to verify that the disposable urine collection device was not used or tampered with prior to its use. The disposable sterile bag 13 will therefore insure the integrity of disposable urine collection device 10.

In an alternative embodiment (not shown), the first open end 12a, and optionally the second open end 12b, of the disposable urine collection device 10 may be covered and temporarily sealed with a removable closure lid that retains the collection bag 14 securely within the tubular body 12 until the disposable urine collection device 10 is ready for use. The closure lid is preferably provided with a tamper-evident indicator that safeguards against tampering with the disposable urine collection device 10 and may be in the form of indicia incorporated to the closure lid for readily indicating tampering with the disposable urine collection device. For added protection against tampering or inadvertent use of the disposable urine collection device 10, the disposable urine collection device provided with the removable closure lids may be stored in a disposable sterile bag as described above.

It will be appreciated that the removable closure lid is optional for the second open end 12b of the disposable urine collection device 10 because the second open end 12b does not provide access into the interior of the tubular body 12 and the collection bag 14 since the collection bag completely encloses the second open end 12b as describe above. On the other hand, the first open end 12a of the disposable urine collection device 10 allows direct access into the tubular body 12 that is configured to receive the male penis during use of the disposable urine collection device 10. The provision of the removable closure lid, with its tamper-evident indicator, to temporarily seal the first open end 12a allows the user, a healthcare professional or any other person handling the disposable urine collection device 10 to verify that it was not previously used or tampered with, thereby insuring against possible infections to the user.

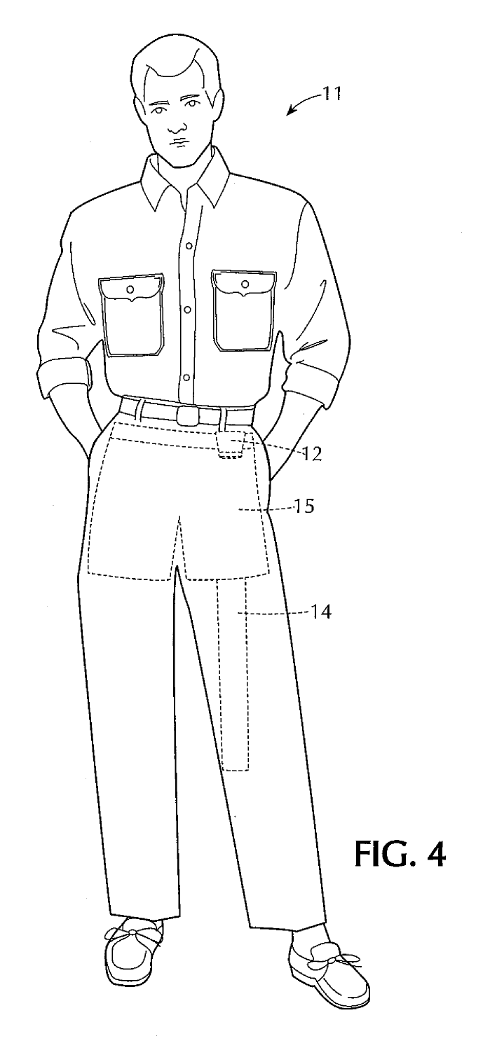

The disposable urine collection device 10 of the present invention may be used while the user is in an erect, squatted, or laying down position. FIG. 4 shows the disposable urine collection device 10 positioned ready for use while the user, generally designated at 11, is in an erect position. In the exemplary embodiment, the disposable urine collection device 10 is discretely positioned inside the user's pants with the tubular body 12 folded over the user's undergarment 15 and with the collection bag 14 in an unfolded state passing through the inside of the undergarment 15 and extending along the user's leg. In this state, the tubular body 12 is slightly compressed (i.e., flattened) so that it conforms better to the space inside the user's pants without creating a visible bulge from the exterior of the pants.

In each of the erect, squatted, or laying down positions, the placement gradient of the disposable urine collection device 10 relative to the user's body is set up such that during urination urine flows down by gravity. The disposable urine collection device 10 can be positioned in place at the time of use, or pre-positioned in place beforehand in the case of a bedridden patient. In use while in the erect position shown in FIG. 4, for example, the user manipulates the tubular body 12 of the disposable urine collection device 10 and inserts the penis into the tubular body through the first open end 12a. In this state, when the user starts urinating, the urine will flow down the tubular body 12 towards the collection bag 14 by gravity by virtue of the placement gradient of the set-up of the disposable urine collection device 10. The urine will easily pass through the tubular body 12, and once the collection bag 14 starts filling with urine, it will sink and seek its own level without competing for the confined space between the user's pants and legs. This is a unique property of the liquid urine that is collected in the collection bag 14, and will leave the user generally comfortable and unaware of its presence no matter how much urine has accumulated inside the collection bag 14.

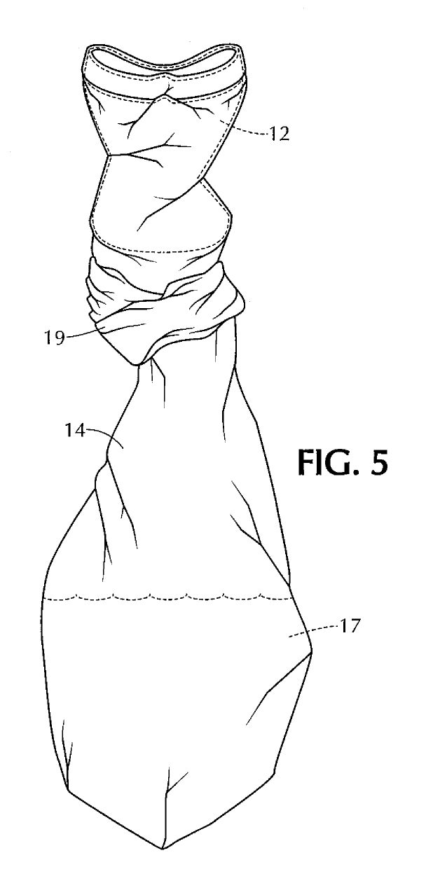

Referring to FIG. 5, when urine 17 is collected in the collection bag 14 after a single use, the tubular body 12 is manually crumpled and the collection bag 14 is tightly sealed by grasping the tubular body with one hand and tying into a knot 19 a portion of the collection bag that does not contain urine 17, e.g., a portion of the collection bag just ahead of the second open end 12b of the tubular body. In this state shown in FIG. 5, the disposable urine collection device 10 is ready to be disposed.

In a modified form of the disposable urine collection device 10 according to the present invention, a pressure relieving member (not shown), such as a piece of mounting tape, is applied over a portion of the exterior peripheral side surface of the tubular body 12 (e.g., directly on the portion of the collection bag 14 covering the exterior surface of the tubular body) proximate the second open end 12b for relieving pressure to the portion of the user's penis that is attached to the pubic bone. A similar type of member is utilized for the female version of the disposable urine collection device 110 described below with reference to FIGS. 6-7, but for a different purpose.

FIGS. 6-7 show another embodiment of the disposable urine collection device, generally designated at 110, according to the present invention. The disposable urine collection device 110 is configured for urination relief by females, with or without bladder control problems. The construction, manner of making, and manner of use of the device 110 is generally the same as for the disposable urine collection device 10 described above for FIGS. 1-5, except as further described below.

One distinction between the disposable urine collection device 10 for males and the disposable urine collection device 110 for females is in the placement orientation of the tubular body which is also preferably in the shape of a cone. As shown in FIGS. 6-7, the orientation of the tubular body, designated by numeral 112, is reversed so that the collection bag, designated by numeral 114, completely encloses the small opening of the cone. The large opening of the cone forming the tubular body 112 remains open so that during use of the disposable urine collection device 110, the large opening of the cone is applied over the female urethra for receiving the urine that is collected by the collection bag 114. That is, in the disposable urine collection device 110, the large opening and small opening of the cone correspond to the first open end 12a and second open end 12b, respectively, of the tubular body 12 in the disposable urine collection device 10 described above with reference to FIGS. 1-5.

It will be appreciated that the conical shape and orientation of the tubular body 112, including its large opening, of the disposable urine collection device 110 allows for the tubular body 112 to function like a funnel which receives the urine flowing from the female urethra and directs and guides the urine towards and into the collection bag 114. However, because of the unique location of the urethra of the female genitalia which is hidden and protected by the labia majora on both sides of the vagina, the space between the two labia of the user's genitalia not completely covered by the large opening of the tubular body 112 and which leads to the anal and buttocks region is a potential source of a possible urine leak. To solve this problem, a plug member 120 is secured to external (via the collection bag 114) and internal surface portions of the tubular body 112 as a means for plugging or blocking the foregoing space between the two labia of the user's genitalia to prevent urine from leaking outside of the tubular body 12 and reaching the anal and buttocks region of the user.

As depicted in FIG. 6, the plug member 120 is formed of a strip of material that is folded over a peripheral edge of the large opening of the tubular body 112 and secured to the external and internal surface portions of the tubular body 112 using suitable securing means, such as adhesive tape 116, for example. The plug member 120 is preferably formed of a soft, pliable foam material. The plug member 120 is formed with a sufficient thickness such that during use of the disposable urine collection device 110, the plug member 120 effectively plugs or blocks the space between the two labia not completely covered by the large opening of the tubular body 112 and which leads to the anal and buttocks region of the user. By this construction, during use the disposable urine collection device 110 effectively receives and directs urine towards the collection bag 114 while preventing the urine from leaking down the anal and buttocks region of the user.

The disposable urine collection device 110 is further provided with a stop member 122 which, as further described below in connection with the disposable urine collection apparatus 210 of FIGS. 8-10, corresponds to means for preventing the disposable urine collection device from inadvertently separating from a diaper-like garment. The stopper member 122 is secured over an exterior peripheral surface portion of the tubular body 112, via the collection bag 114, near the small opening that is completely enclosed by the collection bag. As depicted in FIGS. 6-7, the stop member 122 is formed of two separate pieces of a soft, pliable foam material secured to generally opposite exterior peripheral side surface portions of the tubular body 112 using suitable securing means, such as adhesive tape 116. In an alternative embodiment (not shown), the stop member 122 may be constructed of a single piece of soft, pliable foam material that is secured around the entire circumference of the exterior peripheral side surface portion of the tubular body 112. The stop member 122 is provided with a sufficient thickness (i.e., the protruding distance from the exterior peripheral side surface of the tubular body 112) necessary for it to prevent the tubular body 112 from passing through a slit or opening of the diaper-like garment as described below with reference to the urine collection apparatus 210 shown in FIGS. 8-10. It is understood that the stop member 122 is not necessary for the disposable urine collection device 110 when it is not used in conjunction with the urine collection apparatus 210.

The structure corresponding to the stop member 122 described above in connection with the disposable urine collection device 110 (female version) is also optionally secured on the exterior peripheral side surface of the tubular body 12 of the disposable urine collection device 10 (male version). When applied to the disposable urine collection device 10, the stop member 122 functions as a pressure relieving member that relieve pressure to the portion of the user's penis that is attached to the pubic bone, as described above with reference to the embodiment of FIGS. 1-5.

FIGS. 8-11 show a urine collection apparatus, generally designated at 210, according to an exemplary embodiment of the present invention. The urine collection apparatus 210 contains two components, a diaper-like garment 220 and a disposable urine collection device as described above with reference anyone of the foregoing embodiments shown in FIGS. 1-5 (male version) and 6-7 (female version). FIGS. 8-11 show the urine collection apparatus 210 incorporating the disposable urine collection device 10 (male version) described above with reference to FIGS. 1-5. It is understood, however, that the urine collection apparatus 210 is also suitable for incorporating the disposable urine collection device 110 (female version) described above with reference to FIGS. 6-7.

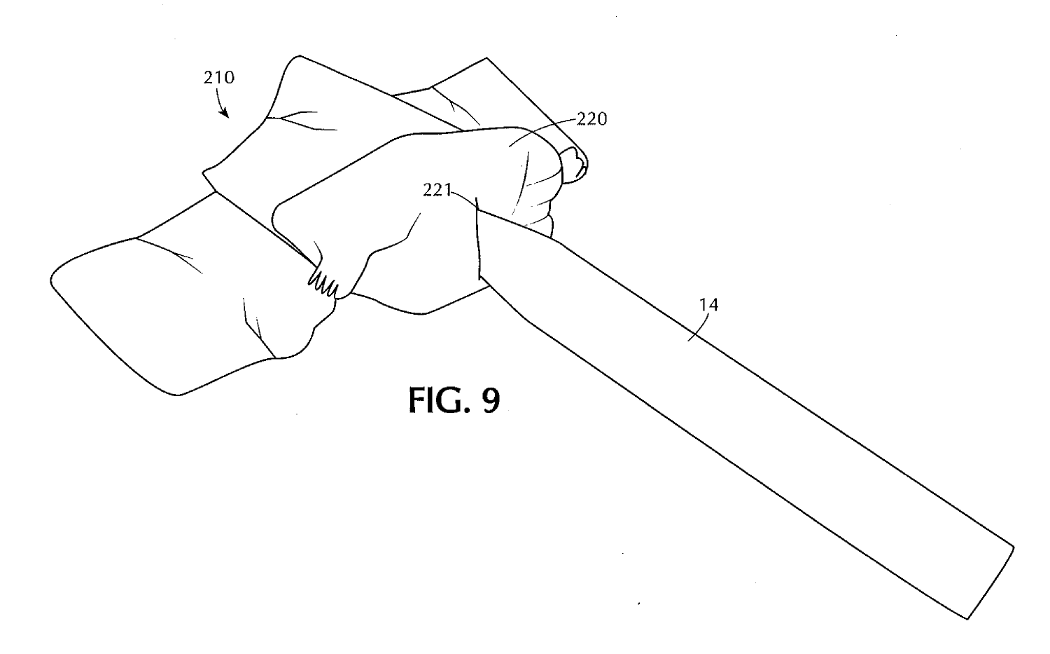

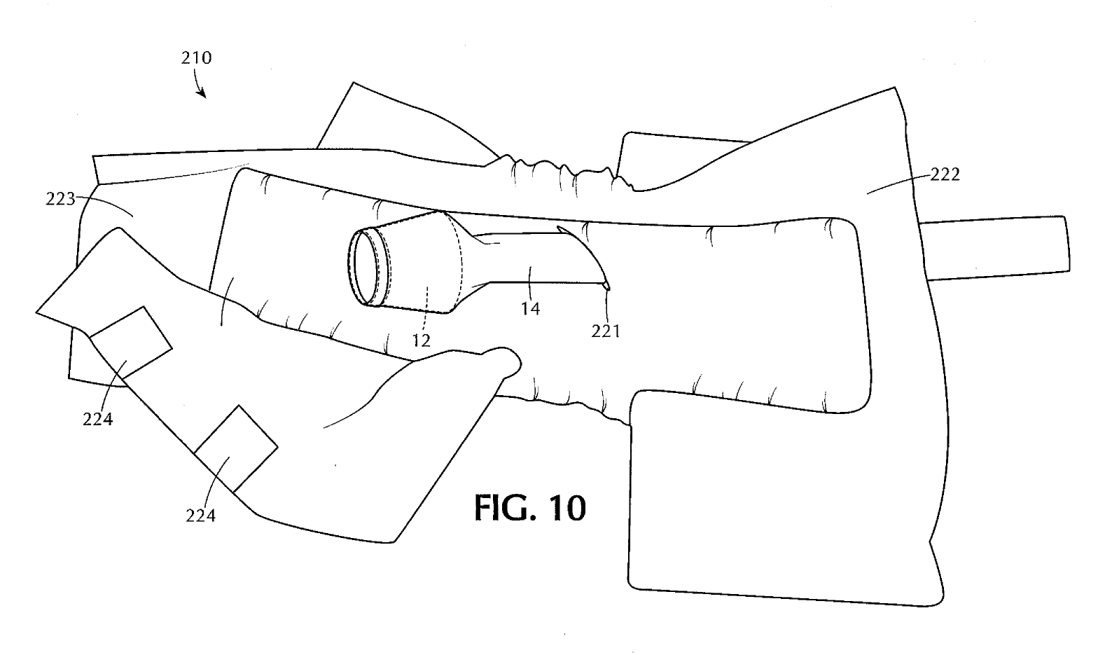

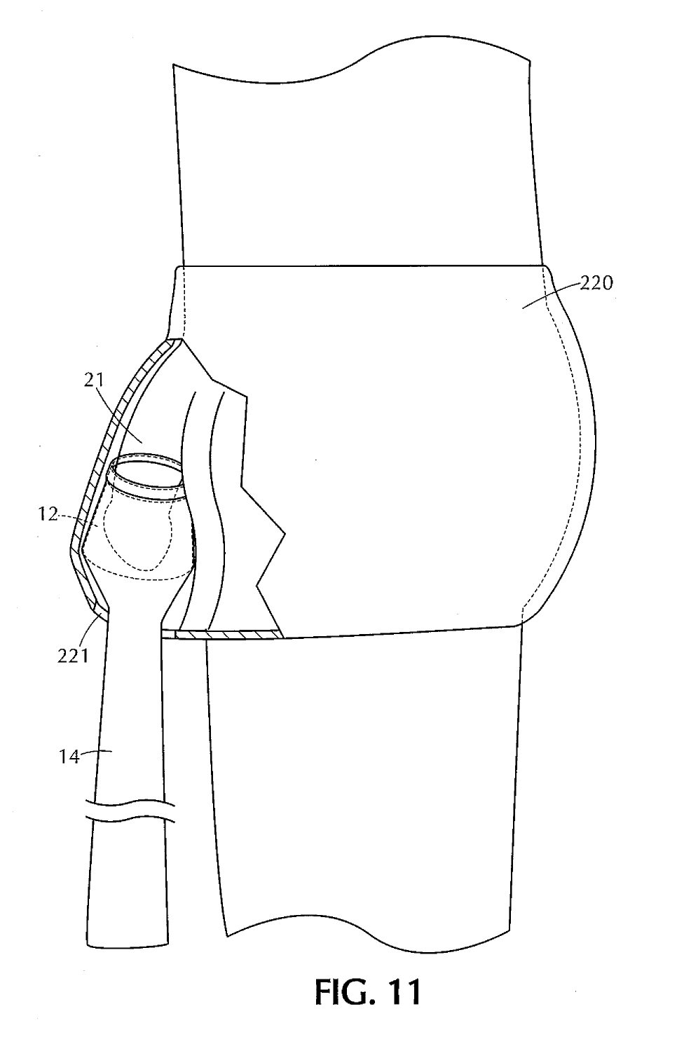

FIG. 8 is a perspective view of the urine collection apparatus 210 showing the diaper-like garment 220 and the disposable urine collection device 10 in an unassembled state. FIG. 9 is a perspective view of the urine collection apparatus 210 showing the diaper-like garment 220 and disposable urine collection device 10 being assembled together in prepare for use. FIG. 10 is a perspective view of the urine collection apparatus 220 showing the diaper-like garment 220 in an open state to illustrate the positioning of the tubular body 12 and portion of the collection bag 14 of the disposable urine collection device 10 relative an inner surface of the diaper-like garment 220. FIG. 11 is a partial side view showing the urine collection apparatus 210 positioned on a user ready for use to collect urine. The urine collection apparatus 210 is particularly suitable for use by patients who are bedridden, paralyzed or handicapped in any setting, including at home, in hospitals, or in nursing homes.

The diaper-like garment 220 is preferably a fitted disposable diaper that is modified with a slit or opening 221 according to the present invention. “Fitted” means that the diaper can be opened completely for easy application and set-up to a patient who is bedridden or handicapped. Such fitted disposable diaper is commercially available and includes, as best shown in FIG. 10, a posterior fold portion 222 configured to protect the back and buttocks region of the user, and an anterior fold portion 223 configured to cover the front and the genitalia of the user. The opening 221 extends through a midline section of the anterior (front) fold portion 223 of the disposable diaper 210. The collection bag 14 is configured to pass through and hang freely outside of the disposable diaper 210, with the tubular body 12 and portion of the collection bag positioned inside of the diaper as shown generally in FIG. 10. The opening 221 is formed with a length (e.g., 4 inches long) sufficient to allow the collection bag 14 to be passed through from the interior to the exterior of the disposable diaper during assembly of the urine collection apparatus 210 as described below.

To assemble the urine collection apparatus 210 for use by a bedridden patient, for example, the disposable diaper 220 is set-up and left open with the posterior (back) fold portion 222 under the buttocks and the anterior (front) fold portion 223 over the genitalia of the patient. The disposable urine collection device 10 is then positioned in place by inserting the patient's penis into the tubular body 12. The collection bag 14 of the disposable urine collection device 10 is then passed through the opening 221, which is located at the midline section of the anterior fold portion 223 generally directly opposite to the location of the user's penis, and positioned outside or to the exterior of the disposable diaper 220. The disposable diaper 220 is then closed in a conventional manner by taping the anterior fold portion 223 to the posterior fold portion 222 at the sides of the hip together using adhesive portions 224 to form a brief-like garment, causing the tubular body 12 to fit snugly inside of the disposable diaper 220 so that the disposable urine collection device 10 is not inadvertently separated from the disposable diaper. Mores specifically, the tubular body 12 enclosing the penis is secured in place from the pressure exerted by the closure of the disposable diaper 220, and the disposable diaper serves to stabilize the tubular body 12 enclosing the penis from getting misplaced or loose.

As described above, the tubular body 12 is in the shape of a cone and is oriented so that it diverges from the first open end 12a to the second open end 12b. The second open end 12b thus corresponds to the large opening of the cone forming a wide mouth with a diameter sufficient to prevent the tubular body 12 from passing through the opening 221 of the disposable diaper 220 (FIG. 10). Thus, the in this embodiment, the second open end 12b of the tubular body constitutes means for preventing the tubular body 12 from passing through the opening 221 of the disposable diaper 220.

In an alternative embodiment (not shown), when the disposable urine collection device 10 is positioned relative to the disposable diaper 220 as shown in FIGS. 10-11, for example, the tubular body 12 may be secured directly to one or more portions of the interior surface of the disposable diaper by additional securing means to further prevent the tubular body 12 from passing through the opening 221 of the disposable diaper 220. For example, the securing means may comprise a double-sided adhesive tape securing the exterior peripheral side surface of the tubular body (e.g., either directly or via the covering collection bag 14) to an interior portion of the disposable diaper 220. Alternatively, the securing means may comprise hook and loop fasteners (e.g., VELCRO® fasteners).

FIG. 11 shows the urine collection apparatus 210 of FIGS. 8-10 in the sate of being worn by a user and ready for collection of urine. The user may be a patient suffering from urine and/or fecal incontinence undergoing care at home or at a medical facility, such as a hospital. FIG. 11 depicts the user in a vertical posture and with the collection bag 14 extending through the opening 221 of the disposable diaper 220 and unfolded downwardly to the exterior of the disposable diaper. It is appreciated that the urine collection apparatus 210 is particularly well-adapted for use by bedridden patients who are unable to urinate while in an erect posture.

In the state of the urine collection device 210 being worn by the patient as shown in FIG. 11, when the patient starts urinating the urine will flow by gravity down the tubular body 12 and into the collection bag 14 hanging outside the disposable diaper 220. The urine will easily pass through the portion of the collection bag 14 passing through the opening 221 of the disposable diaper 220 and collect inside the collection bag 14. When used by bedridden patients, the unfolded portion of the collection bag 14 extending outside of the disposable diaper 220 is sufficiently lowered relative to the tubular body 12 to achieve a placement gradient such that during use the urine easily flows by gravity into the tubular body 12 and downwardly into the collection bag 14. In this manner, once the collection bag 14 starts filling with urine, it will sink to the bed and seek its own level without competing for the confined space between the patient's thighs and buttocks region. This is a unique property of the liquid urine in the collection bag 14.

By the foregoing construction and manner of use of the urine collection apparatus 210 according to the present invention, the patient will be comfortable and unaware of the presence of the urine no matter how much urine has accumulated inside the collection bag 14. The urine collection apparatus 210, by virtue of the collection of the urine by the disposable urine collection device 10, will also keep the patient and the disposable diaper 220 dry and facilitate cleaning of soiled areas, thereby sparing the patient's skin and disposable diaper from getting wet and minimizing the risk of urinary tract infections by isolating the patient's penis inside of the tubular body 12. More specifically, the urine collection device 210 protects the patient's penis and its urethral opening from fecal contamination in patients having fecal incontinence by enclosing the penis inside of the tubular body 12 of the disposable urine collection device 10 and isolating the penis from the surrounding areas of the patient's genitalia.

In an alternative embodiment (not shown), the urine collection apparatus 210 of the present invention is also adapted for use with the disposable urine collection device 110 (female version) described above with reference to FIGS. 6-7. In this embodiment, the stop member 122 secured to the exterior peripheral side surface of the tubular body 112, as described above, preventing the disposable urine collection device 110 from inadvertently separating from the diaper-like garment 220. More specifically, since the orientation of the tubular body 112 is reversed as compared to that of the tubular body 12 of the disposable urine collection device 10, there is a danger that the converging exterior peripheral side surface of the conical-shaped tubular body 112 will cause the tubular body 112 to pass through the opening 221 of the disposable diaper 220 during use such that the large opening of the tubular body 112 is displaced from its position relative to the female's genitalia and prevent all of the urine from being directed into the tubular body and collected by the collection bag 114. The stop member 122 secured to the exterior peripheral side surface of the tubular body 112 functions as a protrusion that effectively increases the diameter of the exterior peripheral side surface of the tubular body 112 on which the stop member 122 is secured to thereby prevent the tubular body 112 from passing through the opening 221 of the disposable diaper 220 and accordingly prevent the disposable urine collection device 110 from inadvertently separating from the disposable diaper during use of the urine collection device. In addition to or alternatively to the use of the stop member 220, the tubular body 112 may be secured directly to one or more portions of the interior surface of the disposable diaper using securing means (e.g., double-sided adhesive tape or hook and loop fasteners) as described above for the urine collection apparatus 210 incorporating the disposable urine collection device 10 shown in FIGS. 8-11.

In the foregoing embodiments of the disposable urine collection devices 10 and 110 according to the present invention, the collection bag is selected with sufficient capacity sufficient to receive the urine output throughout an entire day or night. This would make it very practical for replacement of the disposable urine collection device during preselected work-shifts of the medical providers, such as hospital staff. For example, when used in conjunction with the urine collection apparatus 210, the used disposable urine collection device 10 or 110 is removed by opening the disposable diaper 220 and crumpling the tubular body and passing it through the opening 221 of the disposable diaper. The thus removed disposable urine collection device is then sealed in a manner described above in connection with the disposable urine collection device 10 shown in FIG. 5 for disposal. A new, unused disposable urine collection device 10 or 110 can then be assembled with the disposable diaper 220 ready for use in the manner as described above. It is appreciated that the collection bag of the disposable urine collection device may also be provided with markers or similar indicia to indicate the amount of urine contained inside, particularly during use in hospitals and other medical facilities, for monitoring the urine output.

The present invention also provides for a modified form (not shown) of the disposable urine collection device 10 and 110 according to the present invention that is used, for example, for bedridden patients. This modified form can be used either with device 10 or 110 alone as described above in connection with FIGS. 1-7, or with the urine collection apparatus 210 as described above in connection with FIGS. 8-11. In this modified form of the disposable urine collection device, a peel-off opening is provided at an end or lower portion of the collection bag to accommodate the insertion of a plastic tube that is attached to a separate urine collection bag that hangs by the side of the bed on which the patient rests. This is particularly well-adapted for use as a backup collection bag in cases where for some reason the collection bag of the disposable urine collection device unexpectedly fails to operate properly.

The disposable urine collection device according to the present invention may be conveniently utilized as carry-on disposable urinals by persons with or without bladder control problems who are ambulatory, and not handicapped, so that its use in conjunction with a disposable diaper in the form of the urine collection apparatus as described above is not essential. This is particularly feasible by the fact that the disposable urine collection device is lightweight, discrete, and compact such that it will not occupy a large amount of space nor attract curiosity by people in the vicinity of the user. In the case of tourists, for example, the disposable urine collection device will not attract close scrutiny by airport security. Furthermore, since the disposable urine collection device is disposable (i.e., for one time use only), it is extremely hygienic and particularly handy to carry during travel, especially when the urge to urinate sets in at an awkward situation where immediate relief is inevitable. Individuals using the disposable urine collection device according to the present invention can relieve themselves in any position such as standing, sitting, squatting, or even lying down so long as some degree of privacy is available. Once the user has relieved himself, the used disposable urine collection device can be removed discretely and easily without having to remove the user's garment (e.g., pants, dress). The removed disposable urine collection device is then sealed as described above with reference to FIG. 5 and discarded.

As a carry-on disposable urinal, the disposable urine collection device according to the present invention can be used either in a casual or discrete way. For example, when used in a casual way, the disposable urine collection device 10 (male version) is carried by the user in the compact configuration shown in FIG. 3. When a need to use the disposable urine collection device 10 arises, the user removes the disposable collection device 10 from the disposable sterile bag 13 and unfolds the collection bag which is stored in the interior of the tubular body 12 to place the device 10 in the configuration generally shown in FIG. 1. The user then manipulates the tubular body 12 to position his penis inside the tubular body 12 through the first open end 12a and holds the tubular body in this position, pressing firmly into the surrounding skin, and proceeds to relieve oneself into the tubular body so that the urine is directed and collected inside of the collection bag 14. When done, the user crumples the tubular body 12 and ties a knot 19 on the collection bag 14 as shown in FIG. 5 and then proceeds to discard the used disposable urine collection device 10.

When used in a discrete way, as shown in FIG. 4, for example, the user sets up the carry-on disposable urinal before going out by preferably wearing boxer shorts 15 and by tucking or inserting the carry-on disposable urinal inside the boxer shorts 15 so that the collection bag 14 is unfolded to hang freely along the user's leg corresponding to the user's dominant hand side. The tubular body is pulled up over the garter of the boxer shorts 15 and compressed as shown in FIG. 4 to hold it in place ready for use. The advantage of using the carry-on disposable urinal in this manner is that it is practically weightless and the material used for the tubular body (e.g., STYROFOAM™) does not compete for space or bulge in a confined space because it is soft and easily compressible or pliable yet regains back its shape with slight molding by the hand.

During use in the configuration shown in FIG. 4, the user grabs the tubular body 12 under the pants firmly using the dominant hand and inserts his penis into the tubular body through the first open end 12a while pressing firmly against the skin around it and then starts relieving. Once done, the user crumples the tubular body 12 and twists the collection bag 14 and makes a tying knot 19 as shown in FIG. 5 to close the collection bag containing urine so that it is ready to be discarded. In the event it is inappropriate to dispose of the removed disposable urine collection device 10 right away, the user may simply tuck back the tubular body under the belt of his pants or under and over the garter of the boxer shorts temporarily until the used disposable urine collection device can be disposed of in an appropriate manner.

During urination using the carry-on disposable urinal device either casually or discretely as described above, it would be beneficial to create a distraction, such as by holding a newspaper or blanket or anything in front of the user's pants to camouflage the urination process. A standing position is particularly considered to be the best position for relieving in any situation, but with practice, sitting, squatting and lying positions are also available options especially when the uncontrollable urge to relieve one self sets in.

The female version of the disposable carry-on urinal is also utilized by women in the various situations described above for the male version. For females in particular, the disposable carry-on urinal is particularly well suited for use in situations where the toilet is too filthy to use or sit down and no other options are available. During use, the woman in a standing position with legs spread apart pulls her underwear halfway down and positions the large open end of the tubular body over the female urethra and presses it firmly while relieving. As described above, the tubular body acts as a funnel to catch the urine and direct it towards the collection bag. In this manner, the woman is saved from being made to endure using a filthy toilet bowl.

Thus the disposable urine collection device and urine collection apparatus according to the present invention are very simple to set up that any hospital staff, or even the patient himself if not handicapped, can apply it without difficulty as it does not need to be done utilizing strict sterile techniques and it is externally applied and non-invasive. With a particular application to bedridden patients, the devices and corresponding apparatus according to the present invention can effectively hold the urine output for an entire day or night while maintaining the patient completely dry from the collected urine. The disposable urine collection device and urine collection apparatus according to the present invention can also be easily removed and replaced with new ones in a matter of minutes, and are user friendly, particularly due to their simple construction with no complicated connections or attachments.

By the foregoing description, it will be appreciated that the disposable urine collection device and urine collection apparatus according to the present invention exhibit the following advantages:
Very simple construction, lightweight and handy such that hat it can be carried everywhere by tourists and long distance travelers alike.
Discrete before, during and after use and made from materials that will not attract attention, curiosity or scrutiny by surrounding individuals or by airport security.
Very easy to setup by individuals, particularly by any medical provider including hospital personnel responsible for a patient's personal hygiene.
Very hygienic in that they are disposable and for one time use only.
Protects the penis and its urethral opening from fecal contamination in patients having fecal incontinence by enclosing the penis inside the tubular body, thereby isolating the penis from the surrounding areas of the genitalia.
Minimizes ascending urinary tract infections by isolating the penis inside the tubular body and facilitating cleaning of surrounding soiled areas.
Soft feeling to skin contact in very sensitive areas so that users will hardly notice its presence.
Does not occupy a large amount of space, and the tubular body is compressible yet retains it patency to allow urine to flow through and into the collection bag.

The material for the tubular body (e.g., STYROFOAM®) is compressible yet regains back its shape easily. When crumpled, the material for the tubular body will not form sharp edges that can injure the sensitive skin of the genitalia.

It will also be appreciated from the foregoing description that the following situations, circumstances and uses particularly benefit from the disposable urine collection device and urine collection apparatus according to the present invention:
Well-adapted for long term use by patients with urinary incontinence, including patients who are handicapped or bedridden in hospitals, nursing homes and even at home.

As an emergency carry-on disposable urinal for drivers during long distance travel and passengers or tourists while using public transportation on land, sea or air with limited restroom facilities which often are filthy from overuse.

As a carry-on disposable urinal for construction workers in high rise structures or buildings where toilet facilities are too far, limited and time consuming.

As a carry-on disposable urinal for homeless or displaced people living in public places, especially in cities where they have less opportunities to restroom access.

In times of emergencies or disasters during which displaced people are housed temporarily in closed quarters at temporary shelters or evacuation centers where toilet facilities are limited or filthy from overuse.

As a bedside disposable urinal for hospitalized patients or for elderly people who are prone to accidents and those with limited mobility, whether they suffer from incontinence or not, sparing them from going to the bathroom during the night, thus avoiding potential accidents.

In gathering and events involving large crowds of people, including demonstrations, parades, sporting events, and pilgrimages. In demonstrations particularly, demonstrators sleep in tents where access to bathrooms, whether private or public, is limited.

In the foregoing situations, circumstances and uses, individuals can use the disposable urine collection device and urine collection apparatus according to the present invention when the urge to urinate strikes. This can be accomplished by the individual relieving one self in a sitting, squatting, standing or even lying position with only some limited privacy, and preferably while some minimal type of distraction is created by the individual as described above to avoid generating curiosity by passers-bye.

According to another aspect of the present invention (not shown), in very extreme circumstances or in an emergency for an unrelated purpose, an unused disposable urine collection device according to the present invention can be used to store and carry water.

While the present invention has been described in terms of specific embodiments, it is to be understood that the invention is not limited to these disclosed embodiments. This invention may be embodied in many different forms and should not be construed as limited to the embodiments set forth herein; rather, these embodiments are provided by way of illustration only and so that this disclosure will be thorough, complete and will fully convey the full scope of the invention to those skilled in the art. Indeed, many modifications and other embodiments of the invention will come to mind of those skilled in the art to which this invention pertains, and which are intended to be and are covered by both this disclosure, the drawings and the claims.

1. A disposable urine collection device comprising:
a tubular body having opposite first and second open ends; and
a flexible collection bag for collecting and storing therein urine deposited into the first open end of the tubular body, the collection bag being secured to the tubular body and completely enclosing the second open end of the tubular body to place the first open end in fluid communication with the interior of the collection bag so that urine deposited into the first open end of the tubular body flows into the collection bag for storage therein.

2. A device according to claim 1; wherein the tubular body is made of a compressible foam material.

3. A device according to claim 1; wherein the tubular body is in the shape of a cone having a first end with a first diameter and corresponding to the first open end and having a second end with a second diameter larger than the first diameter and corresponding to the second open end.

4. A device according to claim 1;
wherein the tubular body is in the shape of a cone having a first end with a first diameter and corresponding to the first open end and having a second end with a second diameter smaller than the first diameter and corresponding to the second open end.

5. A device according to claim 1; wherein the collection bag covers at least a portion of an exterior peripheral side surface of the tubular body.

6. A device according to claim 5; wherein the collection bag covers the entire exterior peripheral side surface of the tubular body, is folded over and extends into the first open end, and is secured to an interior surface of the tubular body.

7. A device according to claim 5; wherein the tubular body is in the shape of a cone having a first end with a first diameter and corresponding to the first open end and having a second end with a second diameter larger than the first diameter and corresponding to the second open end.

8. A device according to claim 7; wherein the tubular body is made of a compressible foam material.

9. A device according to claim 5; wherein the tubular body is in the shape of a cone having a first end with a first diameter and corresponding to the first open end and having a second end with a second diameter smaller than the first diameter and corresponding to the second open end.

10. A device according to claim 9; wherein the tubular body is made of a compressible foam material.

11.-20. (canceled)

21. A disposable urine collection device comprising:
a compressible conical-shaped tubular body having opposite open ends; and
a flexible collection bag removably secured to the tubular body so as to completely enclose one of the open ends of the tubular body for collecting and storing therein urine deposited into the other of the open ends of the tubular body during use of the disposable urine collection device.

22. A device according to claim 21; wherein the tubular body is configured to completely store therein the collection bag during non-use of the disposable urine collection device.

23. A device according to claim 21; wherein the one open end of the tubular body has a diameter larger than the other of the open end of the tubular body.

24. A device according to claim 21; wherein the one open end of the tubular body has a diameter smaller than the other of the open end of the tubular body.

25. A device according to claim 21; wherein the collection bag covers at least a portion of an exterior peripheral side surface of the tubular body.

26. A device according to claim 21; wherein the collection bag covers an entire exterior peripheral side surface of the tubular body, is folded over and extends into the other of the open end, and is secured to an interior surface of the tubular body.

27. A device according to claim 21; wherein the tubular body is made of a disposable, liquid-proof compressible material.

28. A device according to claim 27; wherein the material for the tubular body is polystyrene foam.

29. A device according to claim 27; wherein the collection bag is made of a thin, flexible plastic film of material configured to be folded several times to reduce its size to facilitate storage thereof into the tubular body.

30. A device according to claim 21; wherein the collection bag is made of a thin, flexible plastic film of material configured to be folded several times to reduce its size to facilitate storage thereof into the tubular body.
